(19) United States
(12) Patent Application Publication
(10) Pub. No.: US 2025/0250608 A1
(43) Pub. Date: Aug. 7, 2025

(54) METHODS AND COMPOSITIONS FOR ASSAYING BETA-HEXOSAMINIDASE ENZYMATIC ACTIVITY VIA MASS SPECTROMETRY

(71) Applicant: GelbChem, LLC, Seattle, WA (US)

(72) Inventors: Hamid Khaledi, Seattle, WA (US); Michael H. Gelb, Seattle, WA (US); Zackary M. Herbst, Seattle, WA (US)

(73) Assignee: GelbChem, LLC, Seattle, WA (US)

(21) Appl. No.: 18/431,778

(22) Filed: Feb. 2, 2024

(51) Int. Cl. C12Q 1/40

(52) U.S. Cl. CPC C12Q 1/40; G01N 2333/924; G01N 2800/38

(57) ABSTRACT

A method for assaying β-hexosaminidase enzymatic activity, comprising combining a sample with a β-hexosaminidase substrate under conditions sufficient for β-hexosaminidase and the β-hexosaminidase substrate to enzymatically react to produce a β-hexosaminidase enzymatic reaction product; and detecting the β-hexosaminidase enzymatic reaction product via mass spectrometry. The method can be used to diagnose GM2 gangliosidoses and in particular, Tay-Sachs disease and Sandhoff disease. Additionally, reagents for use with the method are herein disclosed.

### I. BACKGROUND

GM2 gangliosidoses are a group of inherited metabolic lysosomal storage disorders caused by the accumulation of glycosphingolipids (GM2 gangliosides) and related glycolipids, glycoproteins, and globosides in neuronal cells. These disorders are characterized by mutations in three genes related to the function of β-hexosaminidase enzymes which catalyze the hydrolysis of the glycosidic linkages of 2-acetamido-2-deoxy-β-d-glycosides. Tay-Sachs disease (TSD) is caused by mutations in the HEXA gene (15q23), Sandhoff disease (SD) is caused by mutations in the HEXB gene (5q13.3), and the third AB variant of GM2 gangliosidosis is caused by mutations in the GM2A gene (5q33.1). To date, 181 HEXA mutations, 103 HEXB mutations, and 9 GM2A mutations have been identified.

The isoenzymes of β-hexosaminidase are dimers composed of α and β subunits, which are encoded by the HEXA and HEXB genes, respectively. The three isoenzymes are β-hexosaminidase A (HexA, a heterodimer of α and β subunits), β-hexosaminidase B (HexB, a homodimer of β subunits), and β-hexosaminidase S (HexS, a homodimer of a subunits). The GM2 activator protein (coded by GM2A) forms a GM2-activator complex as part of the digestion of substrates by the Hex isoenzymes. The presence of both the α and β subunits is required for the binding of the GM2-activator complex to HexA. Following, mutations in the HEXA gene (which codes the α subunit of HexA) may result in loss of HexA enzymatic activity, causing Tay-Sachs disease. Mutations in the HEXB gene, which codes the β subunit of HexA and HexB may result in loss of enzymatic activity of both enzymes, causing Sandhoff disease. The third isoenzyme, HexS, is measurable in small amounts in Sandhoff disease patients, but its activity is not enough to overcome the burden of substrate accumulation.

Cytotoxic effects of the accumulation of GM2 gangliosides occur predominantly in neurons, resulting in a range of symptoms related to neurological impairment, progressive weakness, hypotonia, seizures, and other neurological effects. The phenotypic presentations of the GM2 diseases range from infantile, rapidly progressive severe forms to late-onset, sub-acute, or chronic forms with which patients can survive into adulthood. Typically, diagnosis of afflicted patients begins with the recognition of clinical characteristics, followed by enzymatic activity screening and confirmation with genetic analysis.

With the advent of treatment options for Tay-Sachs disease and Sandhoff disease, early detection thereof is important for optimum clinical response to therapy and correspondingly, may minimize the occurrence of irreversible damage a patient may suffer as a consequence of these diseases. Accordingly, there exists a need for methods of assaying the activity of β-hexosaminidase for screening thereof; the present invention fulfills this need and provides further related advantages.

### II. SUMMARY OF THE INVENTION

The present invention includes a novel method for assaying β-hexosaminidase enzymatic activity, the method including (i) combining a sample with a β-hexosaminidase substrate under conditions sufficient for β-hexosaminidase and the β-hexosaminidase substrate to enzymatically react to produce a β-hexosaminidase enzymatic reaction product, and (ii) detecting the β-hexosaminidase enzymatic reaction product via mass spectrometry. The method can be used to diagnose GM2 gangliosidoses and in particular, Tay-Sachs disease and Sandhoff disease. Additionally, reagents for use with the method are included in the present invention and disclosed herein.

### III. BRIEF DESCRIPTION OF THE DRAWINGS

FIG. 1 is an illustration of β-hexosaminidase, its genes, protein subunits, and isoenzymes.

FIG. 2A is an illustration of a particular embodiment of the novel method for assaying β-hexosaminidase enzymatic activity.

FIG. 2B is an illustration of a particular embodiment of the novel method for assaying β-hexosaminidase A enzymatic activity.

FIG. 2C is an illustration of a particular embodiment of the novel method for assaying β-hexosaminidase A/B enzymatic activity.

FIG. 3 illustrates MRM traces showing peak separation of a HexA substrate, a HexA/B substrate, an internal standard, a HexA enzymatic reaction product, and a HexA/B enzymatic reaction product.

FIG. 4A shows HexA enzymatic reaction product linearity over time as per the novel method. Percent (%) relative error is shown above the data points.

FIG. 4B shows HexA/B enzymatic reaction product linearity over time as per the novel method. Percent (%) relative error is shown above the data points.

FIG. 5A shows a Michaelis-Menten plot for a HexA substrate as per the novel method.

FIG. 5B shows a Michaelis-Menten plot for a HexA/B substrate as per the novel method.

FIG. 6A shows linearity of a HexA enzymatic reaction product as per the novel method.

FIG. 6B shows linearity of a HexA/B enzymatic reaction product as per the novel method.

FIG. 7A shows the lower limit of quantification for a HexA enzymatic reaction product as per the novel method. Percent (%) relative error is shown above the data points.

FIG. 7B shows the lower limit of quantification for a HexA/B enzymatic reaction as per the novel method. Percent (%) relative error is shown above the data points.

FIG. 8A shows cross-laboratory inter-assay precision results for HexA enzymatic activity as per the novel method.

FIG. 8B shows cross-laboratory inter-assay precision results for HexA/B enzymatic activity as per the novel method.

FIG. 9A shows HexA enzymatic activity in patient dried blood spots analyzed at Lab 1 as per the novel method.

FIG. 9B shows HexA/B enzymatic activity in patient dried blood spots analyzed at Lab 1 as per the novel method.

FIG. 10A shows HexA enzymatic activity in patient dried blood spots analyzed at Lab 2 as per the novel method.

FIG. 10B shows HexA/B enzymatic activity in patient dried blood spots analyzed at Lab 2 as per the novel method.

FIG. 11A shows the ratio of HexA/B enzymatic activity over HexA enzymatic activity derived from the data shown in Table 3.

FIG. 11B shows the ratio of HexA/B enzymatic activity over HexA enzymatic activity derived from the data shown in Table 4.

### IV. DETAILED DESCRIPTION OF THE PREFERRED EMBODIMENTS

The present invention provides novel methods for assaying enzymatic activity of β-hexosaminidase. Such methods may be useful for assaying β-hexosaminidase enzymatic activity in individuals, such as newborns, to evaluate whether the individual may be deficient in β-hexosaminidase enzymatic activity and correspondingly (i) may be diagnosed with GM2 gangliosidoses, and (ii) may be a candidate for treatment thereof.

As to particular embodiments, the present invention can provide novel methods for assaying enzymatic activity of β-hexosaminidase which may be (i) practical for high-throughput analysis (such as in a Newborn Screening (NBS) laboratory), and/or (ii) compatible with multiwell plate and multichannel pipetting techniques (or robotics).

In the subsequent description, for the sake of simplicity and brevity, the novel methods will be referred to as “a method” or “the method,” whereby these singular forms are intended to include plural forms, including “at least one,” unless indicated otherwise.

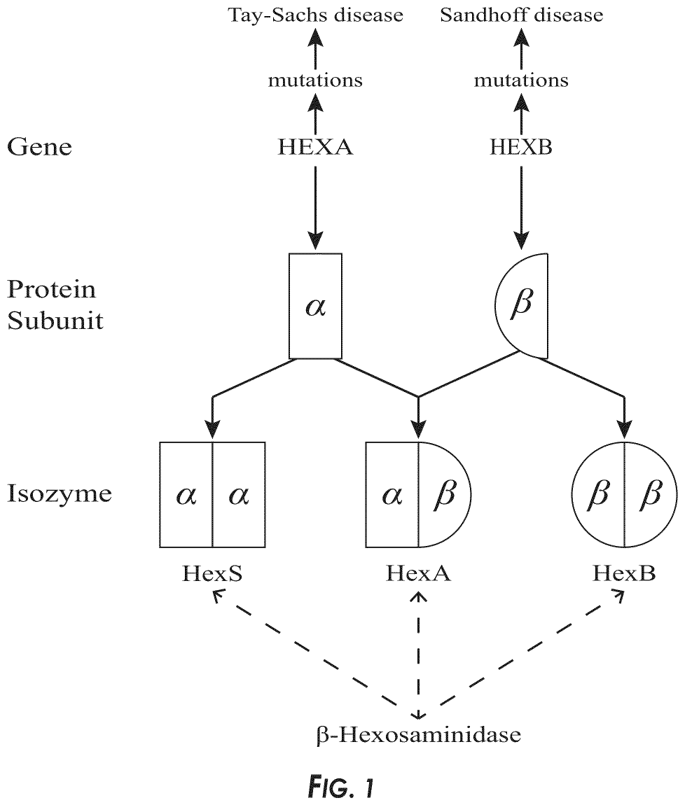

Now referring primarily to FIG. 1, as used herein, the term “β-hexosaminidase” can refer to any one or more of the isoenzymes of β-hexosaminidase, namely β-hexosaminidase A (HexA, a heterodimer of α and β subunits), β-hexosaminidase B (HexB, a homodimer of β subunits), and β-hexosaminidase S (HexS, a homodimer of α subunits).

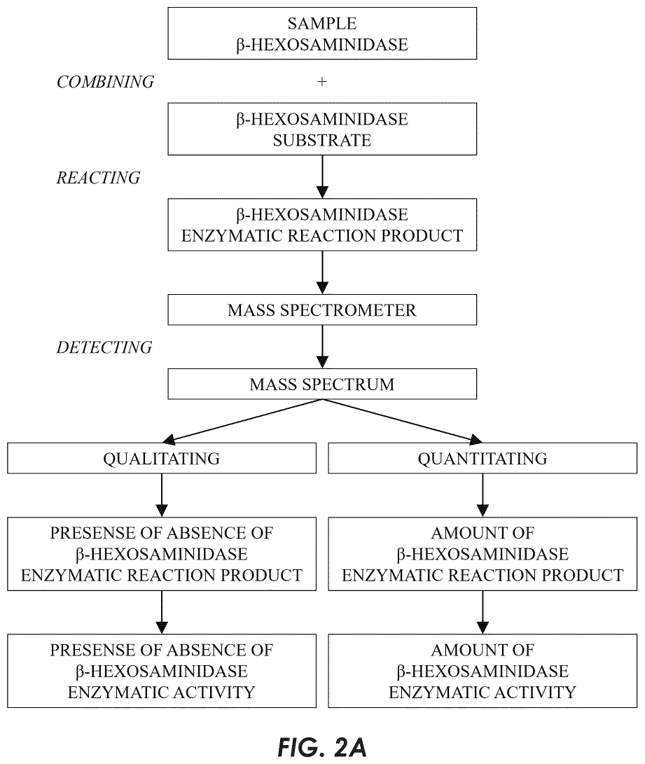

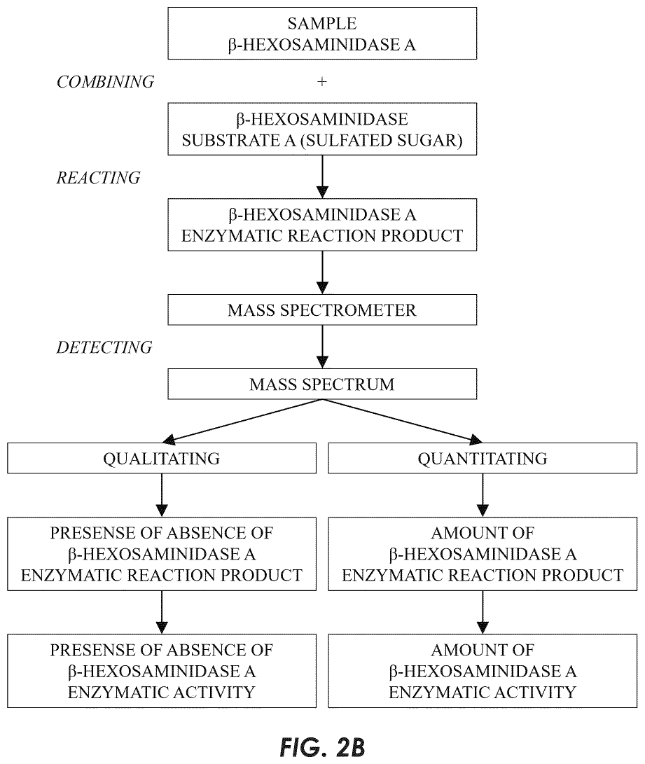

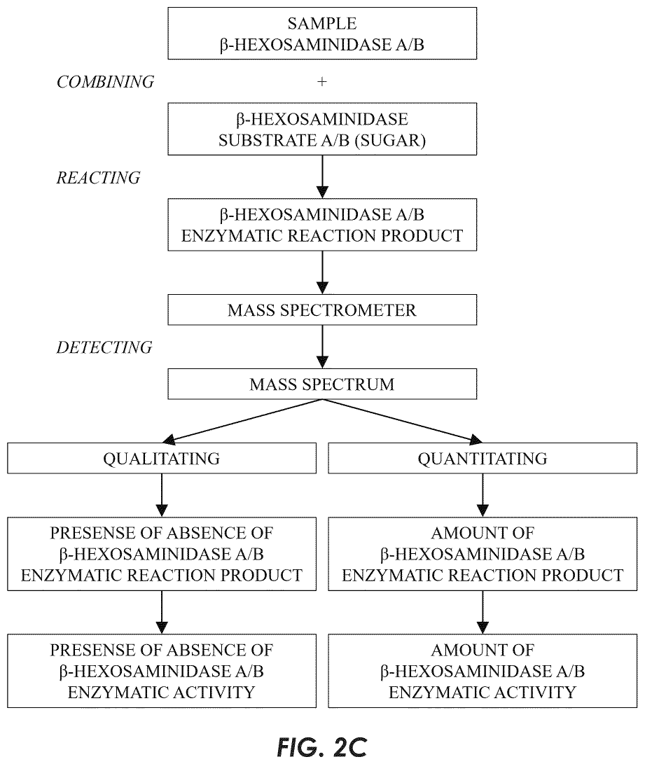

Now referring primarily to FIG. 2A, the activity of an enzyme can be assessed by its ability to enzymatically react with its substrate to produce an enzymatic reaction product. Accordingly, the novel method for assaying enzymatic activity of β-hexosaminidase includes (i) combining a sample with a β-hexosaminidase substrate under conditions sufficient for β-hexosaminidase and the β-hexosaminidase substrate to enzymatically react to produce a β-hexosaminidase enzymatic reaction product, and (ii) detecting the β-hexosaminidase enzymatic reaction product via mass spectrometry.

The method can be performed on any sample that potentially contains β-hexosaminidase, including specimens (e.g., blood, plasma, serum, tissue, cell culture lysate, etc.) from human, animal, or non-living sources (e.g., cell lines, synthetic protein sources, etc.). As to particular embodiments, the sample can be a blood sample. As to particular embodiments, the blood sample can be a dried blood sample. As to particular embodiments, the dried blood sample can be a dried blood spot from an individual, for example but not limited to a newborn in which the dried blood spot may be from a newborn screening card.

To prepare a dried blood spot, blood can be collected and retained on filter paper. As per the method, a portion of the dried blood spot, for example a ˜3 mm punch, can be directly combined with an assay buffer and incubated with a β-hexosaminidase substrate to potentially generate a β-hexosaminidase enzymatic reaction product. Advantageously, the method may not require that the sample be processed before assaying. An additional benefit of the method may be that the sample physical support (e.g., filter paper) may not interfere with assaying, which can be contrary to conventional practice in the art which may typically require that the sample be removed from the sample physical support for assaying.

Again referring primarily to FIG. 2A, the method can include combining or contacting a sample (potentially containing β-hexosaminidase) with an assay buffer to provide a liquid phase (which may or may not be homogeneous). As to particular embodiments, the assay buffer can be an aqueous assay buffer, and this step may be considered an extraction or elution or hydration step in which at least a portion of β-hexosaminidase in the sample can be extracted into an aqueous liquid phase in which an enzymatic reaction between β-hexosaminidase and the β-hexosaminidase substrate may occur.

As to particular embodiments, the aqueous assay buffer can have a pH sufficient to extract β-hexosaminidase from the sample and solubilize β-hexosaminidase, as necessary, to provide an enzymatic reaction mixture in which β-hexosaminidase and the β-hexosaminidase substrate may enzymatically react to produce a β-hexosaminidase enzymatic reaction product. As to particular embodiments, the pH of the aqueous assay buffer can be in a range of between about 2.6 and about 5. As to particular embodiments, the pH of the aqueous assay buffer can be in a range of between about 3 and about 4. As to particular embodiments, the pH of the aqueous assay buffer can be about 3.6. As but one illustrative example of the latter, the aqueous assay buffer can comprise sodium formate buffer (50 mM, pH 3.6) containing sodium taurocholate (2.5 mg/mL). It will be appreciated that in certain embodiments, the sample can be combined with an aqueous assay buffer which includes the β-hexosaminidase substrate.

After combining a sample with a β-hexosaminidase substrate in an aqueous assay buffer, the method can include incubating the resultant enzymatic reaction mixture under conditions sufficient for β-hexosaminidase and the β-hexosaminidase substrate to enzymatically react to produce a β-hexosaminidase enzymatic reaction product. Correspondingly, the enzymatic reaction mixture can be incubated at a pre-determined temperature for a pre-determined amount of time sufficient to produce a β-hexosaminidase enzymatic reaction product. Such temperature and time can vary and will depend, at least in part, on the amount of β-hexosaminidase enzymatic activity within the sample and the sensitivity of the detection method for detecting the β-hexosaminidase enzymatic reaction product. As to particular embodiments, the enzymatic reaction mixture can be incubated at about body temperature or a temperature of about 37° C. for an amount of time in a range of between about 2 hours and about 6 hours. As to particular embodiments, the enzymatic reaction mixture can be incubated at about body temperature or a temperature of about 37° C. for an amount of time in a range of between about 3 hours and about 5 hours. As to particular embodiments, the enzymatic reaction mixture can be incubated at about body temperature or a temperature of about 37° C. for an amount of time of about 4 hours. As to particular embodiments, the enzymatic reaction mixture can be incubated at about body temperature or a temperature of about 37° C. for an amount of time not greater than about 4 hours. As to particular embodiments, the enzymatic reaction mixture can be incubated at about body temperature or a temperature of about 37° C. for an amount of time not greater than about 6 hours. To facilitate the enzymatic reaction, the enzymatic reaction mixture can, but need not necessarily, be shaken during the incubation.

After the pre-determined amount of time, the enzymatic reaction can optionally be quenched (i.e., stopped) by the addition of a quenching agent. As but one illustrative example, the quenching agent can be a solution comprising methanol: ethyl acetate (1:1 v/v).

As to particular embodiments, the present invention can include one or more β-hexosaminidase substrates and one or more β-hexosaminidase enzymatic reaction products useful with the novel method as well as in a variety of assay formats. For example, the present β-hexosaminidase substrate can be detected in an assay designed to analyze β-hexosaminidase substrate consumption during an enzymatic reaction, while the β-hexosaminidase enzymatic reaction product can be detected in an assay designed to analyze its formation during an enzymatic reaction.

Now referring primarily to FIG. 2B, the novel method can include assaying β-hexosaminidase A enzymatic activity, said method including (i) combining a sample with a β-hexosaminidase A substrate under conditions sufficient for β-hexosaminidase A and the β-hexosaminidase A substrate to enzymatically react to produce a β-hexosaminidase A enzymatic reaction product, and (ii) detecting the β-hexosaminidase A enzymatic reaction product via mass spectrometry.

A representative β-hexosaminidase A substrate and corresponding β-hexosaminidase A enzymatic reaction product which may be useful with the novel method are shown in Scheme I below (and available from GelbChem, LLC, Seattle, Washington, USA). The β-hexosaminidase A substrate is specific for β-hexosaminidase A (HexA, a heterodimer of α and β subunits), thus herein referred to as a HexA substrate. Upon enzymatic reaction of HexA with the HexA substrate, a HexA enzymatic reaction product can be produced. Detection of the HexA enzymatic reaction product can provide a measure of HexA enzymatic activity. Detection of the HexA enzymatic reaction product can provide a measure of only HexA enzymatic activity, as HexB does not enzymatically react with the HexA substrate.

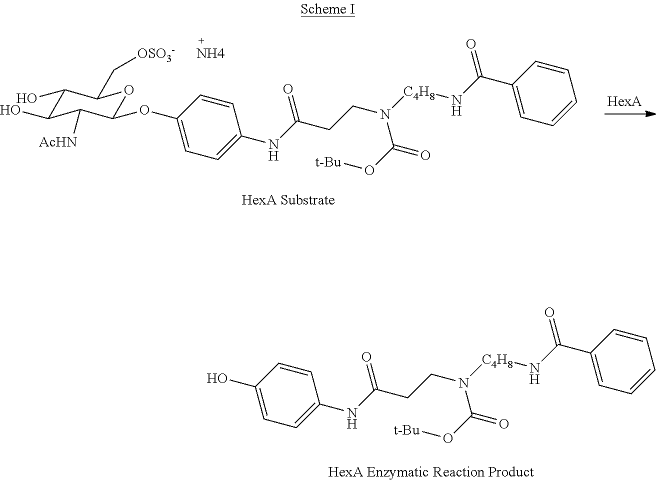

The HexA substrate may be synthetic and similar to the enzyme's natural substrate(s) while at the same time allowing for highly sensitive and specific detection of the resultant HexA enzymatic reaction product via mass spectrometry. Structurally, the HexA substrate can include a sulfated sugar (or carbohydrate) moiety, which may make this substrate specific for only HexA, as HexB does not react with such a sulfated sugar moiety. Accordingly, detection of the resultant HexA enzymatic reaction product can provide a measure of only HexA enzymatic activity.

As illustrative and nonlimiting examples, the sulfated sugar can be sulfated glucose or sulfated galactose, whereby the sugar can be sulfated at any one of positions 2, 3, 4, or 6. Said another way, the sulfated sugar can be a 2-sulfated sugar, a 3-sulfated sugar, a 4-sulfated sugar, or a 6-sulfated sugar.

As to particular embodiments, the sugar can be N-acetylglucosamine (GlcNAc), an amide derivative of the monosaccharide glucose. The GlcNAc can be sulfated at position 6, making this substrate specific for HexA. Incubation with HexA present in a sample can result in the enzymatic release of the HexA substrate's sulfated sugar moiety to produce the HexA enzymatic reaction product. Accordingly, the HexA substrate enables measurement of HexA enzymatic activity, which may typically be low in both Sandhoff disease patients and Tay-Sachs disease patients.

Advantageously, the HexA enzymatic reaction product may easily protonate to improve mass spectrometry sensitivity in positive ion mode. Further, the HexA enzymatic reaction product may be sufficiently hydrophobic for extraction from the aqueous assay buffer into an organic solvent. Moreover, the HexA enzymatic reaction product may undergo a major fragmentation pathway leading to an enhanced signal-to-noise ratio (as opposed to distribution of the signal across multiple product ions).

Now referring primarily to FIG. 2C, the novel method can include assaying β-hexosaminidase A/B enzymatic activity (meaning the total enzymatic activity of both HexA and HexB), said method including (i) combining a sample with a β-hexosaminidase A/B substrate under conditions sufficient for both β-hexosaminidase A and β-hexosaminidase B to enzymatically react with the β-hexosaminidase A/B substrate to produce a β-hexosaminidase A/B enzymatic reaction product, and (ii) detecting the β-hexosaminidase A/B enzymatic reaction product via mass spectrometry.

A representative β-hexosaminidase A/B substrate and corresponding β-hexosaminidase A/B enzymatic reaction product which may be useful with the novel method are shown in Scheme II below (and available from GelbChem, LLC, Seattle, Washington, USA). The β-hexosaminidase A/B substrate is specific for both HexA and β-hexosaminidase B (HexB, a homodimer of β subunits), thus herein referred to as a HexA/B substrate. Upon enzymatic reaction of HexA or HexB with the HexA/B substrate, a HexA/B enzymatic reaction product can be produced. Detection of the HexA/B enzymatic reaction product can provide a measure of the total enzymatic activity of both HexA and HexB.

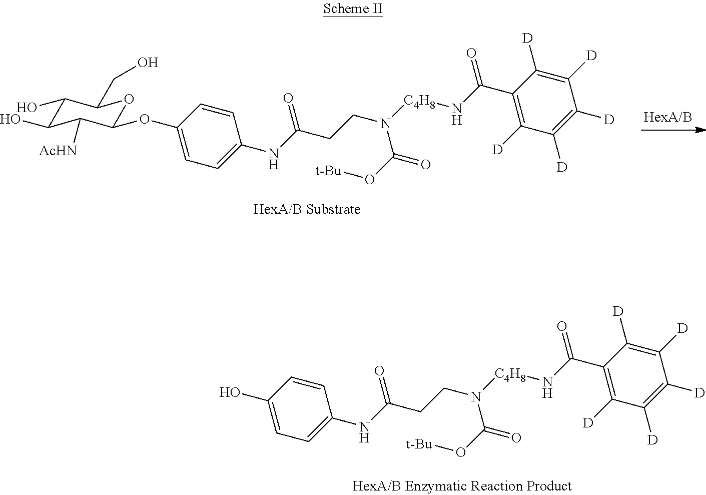

The HexA/B substrate may be synthetic and similar to the enzymes' natural substrates while at the same time allowing for highly sensitive and specific detection of the resultant HexA/B enzymatic reaction product via mass spectrometry. Structurally, the HexA/B substrate can include a non-sulfated sugar (or carbohydrate) moiety, which may make this substrate specific for both HexA and HexB. Accordingly, detection of the resultant HexA/B enzymatic reaction product can provide a measure of total HexA and HexB enzymatic activity.

As illustrative and nonlimiting examples, the non-sulfated sugar can be glucose or galactose.

As to particular embodiments, the sugar can be GlcNAc, a substrate for both HexA and HexA. Incubation with HexA or HexB present in a sample can result in the enzymatic release of the HexA/B substrate's sugar moiety to produce the HexA/B enzymatic reaction product. Accordingly, the HexA/B substrate enables measurement of the total enzymatic activity of both HexA and HexB, which may typically be low in Sandhoff disease patients and normal in Tay-Sachs disease patients.

Advantageously, the HexA/B enzymatic reaction product may easily protonate to improve mass spectrometry sensitivity in positive ion mode. Further, the HexA/B enzymatic reaction product may be sufficiently hydrophobic for extraction from the aqueous assay buffer into an organic solvent. Moreover, the HexA/B enzymatic reaction product may undergo a major fragmentation pathway leading to an enhanced signal-to-noise ratio (as opposed to distribution of the signal across multiple product ions).

The HexA substrate and the HexA/B substrate can each include a sugar (or carbohydrate) moiety attached to a chemical compound having one or more favorable sites for protonation and correspondingly, the compound can be ionizable under mass spectrometry conditions. As an illustrative and nonlimiting example, the compound can be an aglycon, which can include a pair of amides that may provide favorable sites for protonation. Again, as illustrative and nonlimiting examples, the HexA substrate may have Formula I and the HexA/B substrate may have Formula II.

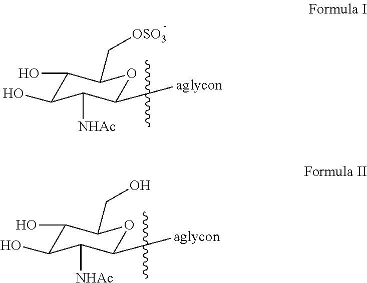

Also as an illustrative and nonlimiting example, the aglycon can have Formula III.

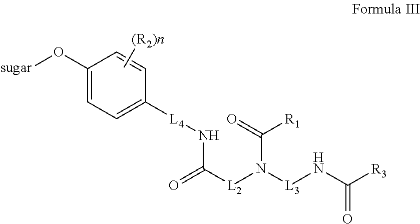

wherein:
L₂ can be a linker comprising 1-20 carbon atoms in which one or more carbon atoms may be replaced with a heteroatom selected from N, O, and S, and/or one or more carbon atoms may be substituted with a C1-C6 alkyl group or halogen;
L₃ can be a linker comprising 1-20 carbon atoms in which one or more carbon atoms may be replaced with a heteroatom selected from N, O, or S, and/or one or more carbon atoms may be substituted with a C1-C6 alkyl group or halogen;
L₄ may be optional; when present, L₄ can be a linker comprising 1-20 carbon atoms in which one or more carbon atoms may be replaced with a heteroatom selected from N, O, or S, and/or one or more of carbon atoms may be substituted with a C1-C6 alkyl group or halogen;
R₁ can be a C1-C10 alkyl group or a C1-C10 alkoxy group;
R₂ at each occurrence can be independently selected from a C1-C10 alkyl group, a C1-C10 alkoxy group, halogen, nitro, —C(═O)NHR, or —C(═O)OR, where R is a C1-C8 alkyl group;
R₃ can be a C1-C10 alkyl group or a substituted or unsubstituted C6-C10 aryl group;
n can be 0, 1, 2, 3, or 4; and
the sugar can be the sugar shown in Formula I or Formula II.

Notably, neither the HexA substrate nor the HexA/B substrate of the present invention include a fluorophore moiety, which may be contrary to conventional β-hexosaminidase assays in the art which can typically be fluorometric assays.

The HexA substrate and the HexA/B substrate shown above in Schemes I and II, respectively, can have identical chemical structures (except for the sulfation in the HexA substrate) but be isotopically different. For example, the HexA/B substrate can be a stable isotope-labeled analog of the HexA substrate in which one or more atoms are replaced by corresponding atomic isotopes so as to generate a detectable mass difference between the resultant HexA enzymatic reaction product and the HexA/B enzymatic reaction product which is distinguishable via mass spectrometry.

As to particular embodiments, the HexA/B substrate can include deuterium in place of one or more hydrogens for selective detection of the resultant HexA/B enzymatic reaction product relative to the HexA enzymatic reaction product via mass spectrometry, as the deuterium-containing HexA/B enzymatic reaction product can be “heavier” than the HexA enzymatic reaction product. Of course, it is herein contemplated that the HexA/B substrate and the HexA substrate can be isotopically labeled in reverse such that the HexA substrate can be a stable isotope-labeled analog of the HexA/B substrate. Additionally, use of other heavy isotopes for substitution to generate mass differentiated enzymatic reaction products is herein contemplated, for example use of carbon-13, nitrogen-15, etc.

When the HexA enzymatic reaction product and the HexA/B enzymatic reaction product are analyzed by mass spectrometry, the resulting mass spectrum reveals a spatial separation thereof, each represented by its own peak (or unique mass spectrometry signature). The relative amounts of the HexA enzymatic reaction product and the HexA/B enzymatic reaction product can be reflected by peak intensity (such as peak area) at each's known mass-to-charge ratio (m/z).

As to particular embodiments, the method can be qualitative and determine the presence or absence of β-hexosaminidase enzymatic activity in a sample by detecting the presence or absence of the corresponding β-hexosaminidase enzymatic reaction product. The presence of the β-hexosaminidase enzymatic reaction product can indicate the presence of corresponding β-hexosaminidase enzymatic activity in the sample, and the absence of the β-hexosaminidase enzymatic reaction product can indicate the absence of corresponding β-hexosaminidase enzymatic activity in the sample. Of note, a qualitative method that determines the presence or absence of a β-hexosaminidase enzymatic reaction product may not require the use of an internal standard.

As to other particular embodiments, the method can be quantitative and determine not only the presence or absence of a β-hexosaminidase enzymatic reaction product, but also the amount of the β-hexosaminidase enzymatic reaction product. As to particular embodiments, the amount of the β-hexosaminidase enzymatic reaction product can be determined by comparing a signal derived from the β-hexosaminidase enzymatic reaction product to a signal derived from a known quantity of an internal standard. As the amount of the β-hexosaminidase enzymatic reaction product is determined by the enzymatic activity of β-hexosaminidase on a β-hexosaminidase substrate that is added to a sample to be assayed, quantification of the β-hexosaminidase enzymatic reaction product provides a measure of the β-hexosaminidase enzymatic activity in the sample.

As to particular embodiments, the internal standard can be related to the β-hexosaminidase enzymatic reaction product. As to particular embodiments, the internal standard and the β-hexosaminidase enzymatic reaction product can have identical chemical structures but be isotopically different. For example, the internal standard can be a stable isotope-labeled analog of the β-hexosaminidase enzymatic reaction product in which one or more atoms are replaced by corresponding atomic isotopes so as to generate a detectable mass difference between the internal standard and the β-hexosaminidase enzymatic reaction product which is distinguishable via mass spectrometry and allows for quantitation thereof. An illustrative example of such an internal standard which is a stable isotope-labeled analog the β-hexosaminidase A enzymatic reaction product and the β-hexosaminidase A/B enzymatic reaction product detailed above is shown in Formula IV.

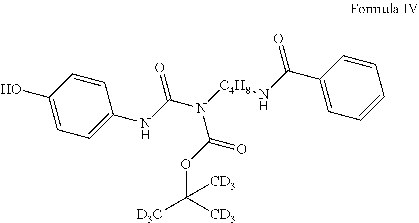

As to particular embodiments, the internal standard can include deuterium in place of one or more hydrogens for selective detection relative to the β-hexosaminidase enzymatic reaction product via mass spectrometry, as the deuterium-containing internal standard can be “heavier” than the β-hexosaminidase enzymatic reaction product. Of course, it is herein contemplated that the internal standard and β-hexosaminidase substrate (which produces the β-hexosaminidase enzymatic reaction product) can be isotopically labeled in reverse such that the β-hexosaminidase substrate can be a stable isotope-labeled analog of the internal standard, thereby producing a stable isotope-labeled β-hexosaminidase enzymatic reaction product. Additionally, use of other heavy isotopes for substitution to generate mass differentiated internal standards and β-hexosaminidase enzymatic reaction products is herein contemplated, for example use of carbon-13, nitrogen-15, etc.

When the internal standard and β-hexosaminidase enzymatic reaction product are analyzed by mass spectrometry, the resulting mass spectrum (plot that shows the intensity of ions based on their m/z) reveals a spatial separation thereof, each represented by its own peak (or unique mass spectrometry signature). The known amount of internal standard can be reflected by peak intensity at its known m/z. The amount of β-hexosaminidase enzymatic reaction product can be determined by comparison of peak intensity at its known m/z relative to the peak intensity of the internal standard.

Quantification of an analyte can be extremely accurate when the analyte and the internal standard have identical chemical structures but are isotopically different. Employment of such an internal standard can account for loss of enzymatic reaction product due to sample handling and/or chemical changes. Additionally, the identical structures of the analyte and internal standard can allow for signal correction due to changes in ionization suppression in the electrospray source. Such suppression may be caused by non-analyte components of a sample that coelute with the analyte of interest, which may be nearly inevitable with a complex biological sample, such as a dried blood spot.

Of note, the internal standard can be introduced at different points in the method. As to particular embodiments, the internal standard can be added prior to incubation with a sample and before, after, or simultaneously with the addition of the β-hexosaminidase substrate. As to other particular embodiments, the internal standard can be added during incubation of the sample and the β-hexosaminidase substrate. As to other particular embodiments, the internal standard can be added during quenching of the enzymatic reaction. As to other particular embodiments, the internal standard can be added to the quenched enzymatic reaction mixture that includes the enzymatic reaction product(s). As to other particular embodiments, the internal standard can be added after sample workup and before mass spectrometry analysis.

As to particular embodiments, prior to mass spectrometry analysis, the method can include separating the β-hexosaminidase enzymatic reaction product and internal standard (if using) from the aqueous enzymatic reaction mixture (and thus, from at least some β-hexosaminidase and/or at least some excess β-hexosaminidase substrate), for example following quenching of the enzymatic reaction. As to particular embodiments, the method can include extracting the β-hexosaminidase enzymatic reaction product and internal standard (if using) from the aqueous enzymatic reaction mixture via an organic solvent to provide an organic phase comprising the β-hexosaminidase enzymatic reaction product and internal standard (if using) for further analysis. Suitable organic solvents can be substantially immiscible with water and may not be effective in solubilizing β-hexosaminidase and/or excess β-hexosaminidase substrate. Additionally, suitable organic solvents can selectively and efficiently extract the β-hexosaminidase enzymatic reaction product and internal standard (if using), and extract each substantially equally (i.e., the β-hexosaminidase enzymatic reaction product and internal standard (if using) can have substantially the same partition coefficient for a given solvent).

As to particular embodiments, suitable organic solvents can include ethyl acetate, diethyl ether, chloroform, methylene chloride, butanol, or the like. As to particular embodiments, the organic solvent can be ethyl acetate. As to particular embodiments, the β-hexosaminidase enzymatic reaction product and internal standard (if using) can be extracted with ethyl acetate and sodium chloride solution for further analysis.

Now referring primarily to FIGS. 2A through 2C, the method can further include (i) detecting or (ii) determining the quantity of the β-hexosaminidase enzymatic reaction product in a sample via mass spectrometry. As an illustrative example, a suitable mass spectrometry technique can be tandem mass spectrometry (MS/MS) performed on a tandem mass spectrometer. For MS/MS analysis, the β-hexosaminidase enzymatic reaction product can be ionized to generate a precursor (or parent) ion of a specific m/z. The precursor ion can then be fragmented (typically via collision-induced dissociation) to provide a fragment ion for analysis. Determining the quantity of the β-hexosaminidase enzymatic reaction product via its fragment ion can be done with use of an internal standard and peak intensity comparisons, as described above. Following, the quantity of the β-hexosaminidase enzymatic reaction product can be used to determine whether the sample is from an afflicted individual who may be a candidate for treatment of GM2 gangliosidoses.

As an illustrative example, mass spectrometry can be carried out on a triple-quadrupole mass spectrometer operating in positive-ion, multiple-reaction monitoring mode. The precursor ions for the HexA enzymatic reaction product, the HexA/B enzymatic reaction product, and the internal standard (m/z 456.5, 461.5, and 465.5, respectively) can be isolated and subjected to collision-induced dissociation to produce corresponding fragment ions (m/z 356.1, 361.2, and 357.1, respectively) for analysis.

As to particular embodiments, liquid chromatography can be employed for introduction of a sample into the mass spectrometer.

As to particular embodiments, the novel method can include assaying enzymatic activity of HexA, which may include quantitation via use of an internal standard.

As to particular embodiments, the method can include assaying enzymatic activity of HexA and HexB, which may include quantitation via use of an internal standard.

As to particular embodiments, the method can be a duplex (or multiplex) assay and correspondingly can comprise simultaneously assaying enzymatic activity of HexA and HexA/B, which may include quantitation via use of an internal standard. As the HexA enzymatic reaction product and the HexA/B enzymatic reaction product shown in Schemes I and II, respectively, can have identical chemical structures but unique mass spectrometry signatures due to isotopic differentiation, a single internal standard (such as that shown in Formula IV) can (i) be used for quantification of the HexA enzymatic reaction product and the HexA/B enzymatic reaction product, derived from a single well of a multiwell plate, in a single analytical run (a single injection into the mass spectrometer), and (ii) avoid isobaric interference therebetween. Such a duplex HexA and HexA/B enzymatic activity assay can save time and resources, for example in the high-throughput screening setting of a NBS laboratory, where multiplexability with other enzyme activity assays may be advantageous.

The HexA and HexA/B duplex enzymatic activity assay disclosed herein may be superior to fluorometric methods for measuring HexA and HexA/B enzymatic activity, which require separate incubations and measurements. Further, the HexA and HexA/B duplex enzymatic activity assay disclosed herein may be superior to a reported mass spectrometric enzyme assay which also requires separate incubations as well as a heat inactivation step (B. Fitterer, P. Hall, N. Antonishyn, R. Desikan, M. Gelb, D. Lehotay, Incidence and carrier frequency of Sandhoff disease in Saskatchewan determined using a novel substrate with detection by tandem mass spectrometry and molecular genetic analysis Mol Genet Metab 111 (2014) 382-389). Beneficially, the HexA and HexA/B duplex enzymatic activity assay disclosed herein eliminates the need for multiple incubations and heat inactivation, and introduces the option to multiplex with other enzyme activity assays as part of an efficient screening panel in NBS laboratories. This degree of multiplexing is not possible with fluorometric assays.

Now regarding a clinical application, the novel method can be used to screen individuals for a deficiency in β-hexosaminidase enzymatic activity. Further, the novel method can be used to screen individuals for a deficiency in HexA enzymatic activity. Still further, the novel method can be used to screen individuals for a deficiency in HexA/B enzymatic activity. Still further, the novel method can be used to screen individuals for a deficiency in both HexA enzymatic activity and HexA/B enzymatic activity.

Accordingly, the novel method can be used to screen individuals for Tay-Sachs disease. Further, the novel method can be used to screen individuals for Sandhoff disease. Still further, the novel method can be used to screen individuals for both Tay-Sachs disease and Sandhoff disease.

As to particular embodiments, the novel method can be used to screen newborns.

As to particular embodiments, the novel method can be incorporated into a kit, such as a diagnostic kit for assaying β-hexosaminidase enzymatic activity. As to particular embodiments, the diagnostic kit may be useful with dried blood spot samples.

The kit can include one or more of a β-hexosaminidase substrate, an internal standard, a control (such as control dried blood spots, a β-hexosaminidase enzymatic reaction product, etc.), assay buffer, a multiwell plate (possibly coated with the β-hexosaminidase substrate), instructions for use of the kit, etc., or the like.

### EXAMPLES

### Example 1: Preparation of Duplex Assay Cocktail

A duplex assay cocktail for use with the novel method to simultaneously assay HexA enzymatic activity and HexA/B enzymatic activity was prepared by combining methanolic solutions of the HexA substrate and the HexA/B substrate and the internal standard of the novel method (all available from GelbChem, LLC, Seattle, Washington, USA) and removing the solvent under vacuum. The dried solids were reconstituted in sodium formate buffer (50 mM, pH 3.6) containing sodium taurocholate (2.5 mg/mL). Final concentrations of each reagent were as follows: HexA substrate (1.0 mM), HexA/B substrate (250 μM), and internal standard (5 μM). The duplex assay cocktail was prepared fresh before each assay. Methanolic solutions were stored at −20° C. in TEFLON® septum capped glass vials with caps tightly wrapped in PARAFILM®.

### Example 2: Duplex Assay Method for Analysis of Dried Blood Sample

3.2 mm punches were taken from dried blood samples. The punches were added into a well of a multiwell plate and in particular, a 1.0 mL deep-well 96-well plate. Following, 30 μL of the duplex assay cocktail described above was added to each well. The plate was sealed with a silicone sealing mat and shaken at 250 rpm for 4 hours at 37° C. in an orbital shaker incubator. After incubation, the enzymatic reaction was quenched by adding 100 μL of methanol: ethyl acetate (1:1) and mixed by pipetting up and down ˜10 times. Analytes were extracted by adding sodium chloride solution (0.5 M, 200 μL) and ethyl acetate (400 μL) to each well and mixed by pipetting up and down ˜10 times. The plate was centrifuged at 3000×g for 5 min at ambient temperature, then 200 μL of the upper ethyl acetate layer was transferred to a 96-well microplate. The ethyl acetate was evaporated from each well using a nitrogen jet at ambient temperature, then each well was reconstituted with 100 μL methanol:water (1:1). The plate was covered with aluminum foil prior to UPLC-MS/MS analysis.

Analytes were separated via a WATERS™ ACQUITY 2D Binary Solvent UPLC system fitted with a WATERS™ ACQUITY UPLC CSH C18 column, 130 Å, 1.7 μm, 2.1 mm×50 mm (SKU: 186005296) and a WATERS™ ACQUITY UPLC CSH C18 VanGuard pre-column, 130 Å, 1.7 μm, 2.1 mm×5 mm (SKU: 186005303). The columns were held at 55° C. The mobile phases were 0.1% formic acid in water (solvent A) and 0.1% formic acid in acetonitrile (solvent B). All solvents were LC-MS Optima Grade from Fisher Scientific. Analytes were eluted through a gradient solvent program with a constant flow rate of 0.6 mL/min. Gradient starting composition was 20% solvent B with linear gradient to 45% B (0.00-0.50 min), held at 45% B (0.50-0.60 min), gradient to 20% B (0.60-0.90 min), gradient to 75% B (0.90-1.40 min), gradient to 100% B (1.40-1.41 min), held at 100% B (1.41-1.60 min), gradient to 20% B (1.60-1.61 min), then finally held at 20% B (1.61-1.80 min) to re-equilibrate. To minimize contamination in the source, flow was diverted to waste prior to 0.80 min and after 1.20 min. Sample injection volume was 10 μL and the sample manager loop was set to offline for 0.5 min.

Analytes were detected via a WATERS™ Xevo TQ triple-quadrupole mass spectrometer. The mass spectrometer parameters were set as follows: source temperature 150° C., capillary 3.5 kV, desolvation temperature 500° C., desolvation gas 1,000 L/Hr, cone gas 30 L/Hr, and collision argon gas 0.15 mL/min. MS/MS data was collected using a multiple reaction monitoring (MRM) mode between 0.80-1.20 min. The following MRM traces were monitored for quantitation: HexA enzymatic reaction product (456.5>356.1), HexA/B enzymatic reaction product (461.5>361.2), and internal standard (465.5>357.1). The MRM traces showing peak separation of HexA substrate, HexA/B substrate, internal standard, HexA enzymatic reaction product, and HexA/B enzymatic reaction product are shown in FIG. 3.

### Example 3: Enzyme Kinetics

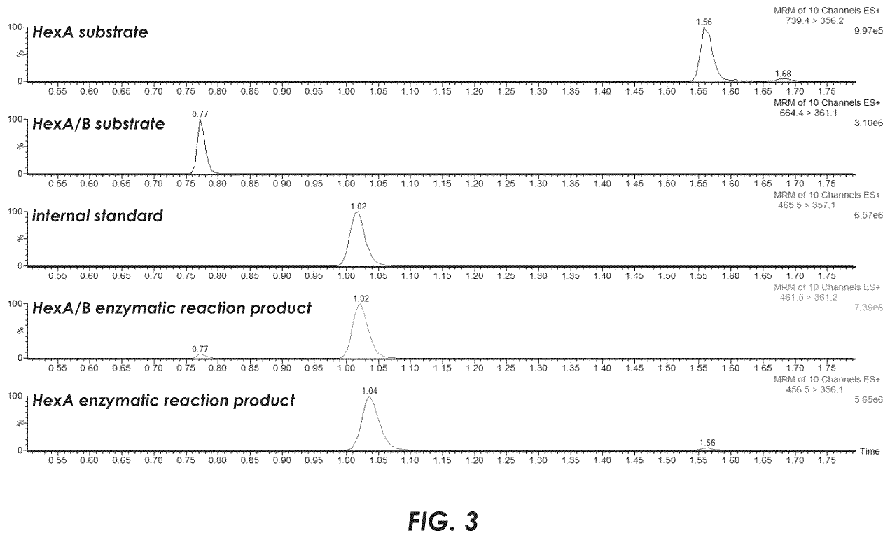

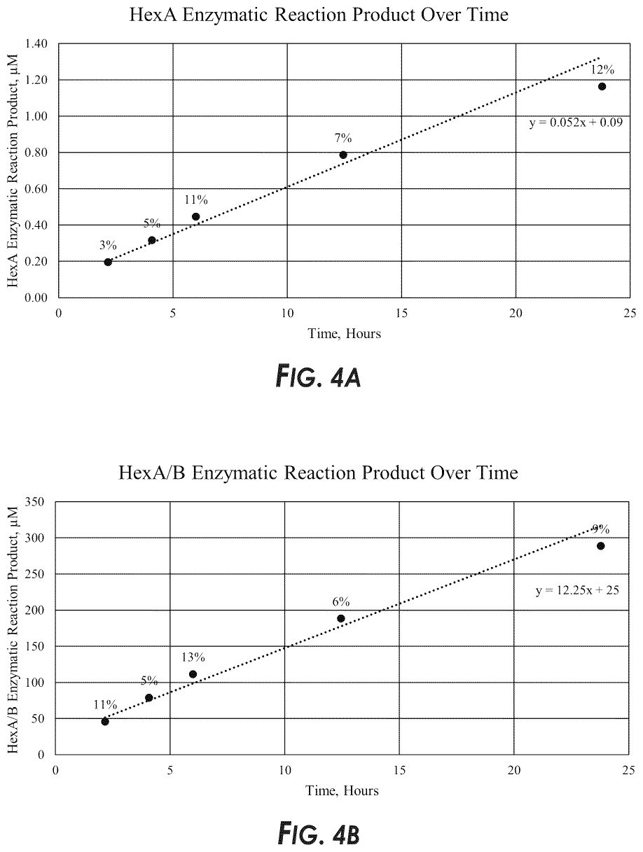

Enzyme kinetics of HexA and HexA/B were measured in relationship to the HexA substrate and HexA/B substrate, respectively, using endogenous enzymes from healthy adult dried blood spots. The production of both the HexA enzymatic reaction product and the HexA/B enzymatic reaction product over time was determined to be constant by a linear increase in the respective enzymatic reaction product concentration over 24 hours (% relative error of line fit for each timepoint is <15%). FIG. 4A shows the HexA enzymatic reaction product linearity over time, and FIG. 4B shows the HexA/B enzymatic reaction product linearity over time.

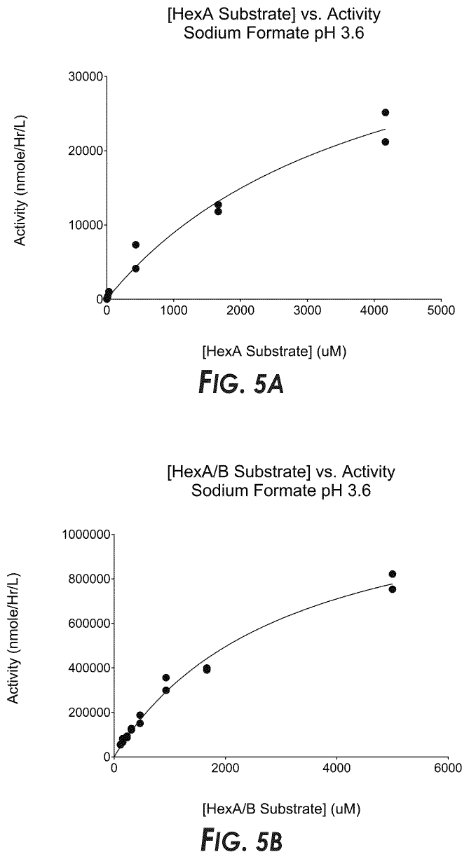

The HexA substrate and HexA/B substrate were stable over a 24 hour incubation at 37° C. in sodium formate buffer, with over 99.99% recovery of the substrates. The reaction rate for each substrate at different substrate concentrations was measured and the relationship found to be hyperbolic. From this data, the Vmax was determined to be 45,188 for the HexA substrate and 1.250e+006 for the HexA/B substrate. Further, the Michaelis-Menten constants (Km) were determined to be 4,051 μM for the HexA substrate and 3,063 μM for the HexA/B substrate. FIG. 5A shows the Michaelis-Menten plot for the HexA substrate, and FIG. 5B shows the Michaelis-Menten plot for the HexA/B substrate.

### Example 4: Competitive Inhibition

The HexA/B substrate binds to both HexA and HexB, whereas the HexA substrate binds to only the HexA; thus, the HexA/B substrate may theoretically block the action of HexA on the HexA substrate by competitive binding (i.e. competitive inhibition). An experiment was done to test this theory where HexA enzymatic activity was measured in dried blood spots from healthy individuals using the HexA substrate in the presence and absence of the HexA/B substrate. From this experiment, it was determined that there is no competitive inhibition of HexA enzymatic activity under the experimental conditions.

### Example 5: Substrate Working Concentrations

Final working concentrations of the HexA substrate and the HexA/B substrate were chosen based on the amount of corresponding enzymatic reaction product produced after a 4 hour incubation with one dried blood spot punch. It was determined that 1 mM of HexA substrate was required to produce a quantifiable amount of HexA enzymatic reaction product for analysis by LC-MS/MS. Due to higher specific activity, 250 μM of HexA/B substrate was sufficient to produce a quantifiable amount of HexA/B enzymatic reaction product for analysis by LC-MS/MS.

### Example 6: Analytical Range, Repeatability, and Enzyme Stability

The analytical range, as calculated by dividing the enzymatic activity in healthy individual samples over the enzymatic activity in a blank, was determined to be 82 for the HexA substrate and 3,400 for the HexA/B substrate. Repeatability was determined by same-day analysis of eight replicate incubations from a healthy adult dried blood spot and was found to have a % coefficient of variation (% CV) of 8.7%. The stability of HexA and HexB in dried blood spots has been previously studied and both enzymes were found to be stable after storage at room temperature for up to 38 months.

### Example 7: Analytical Verification of Assay

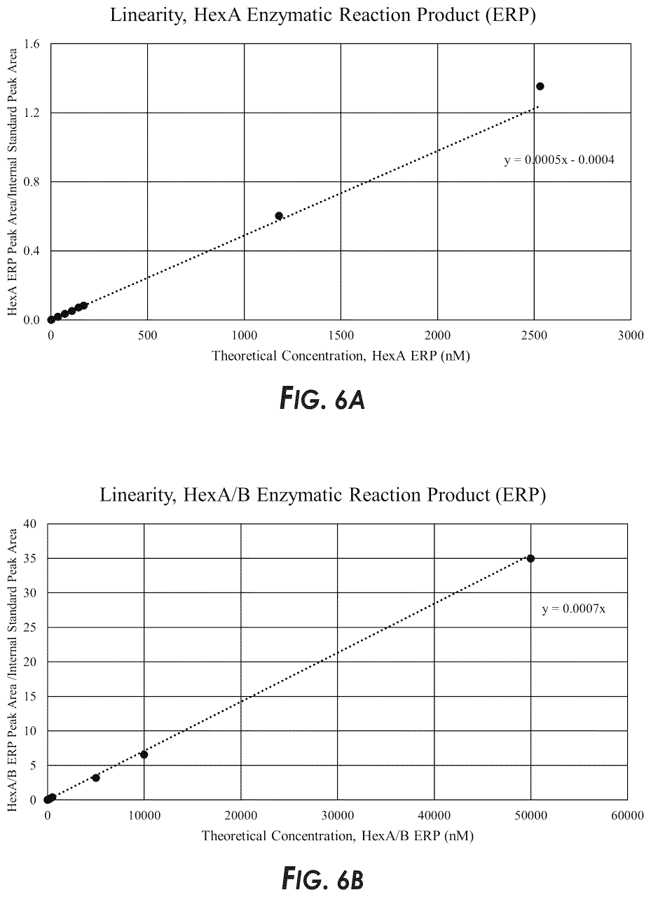

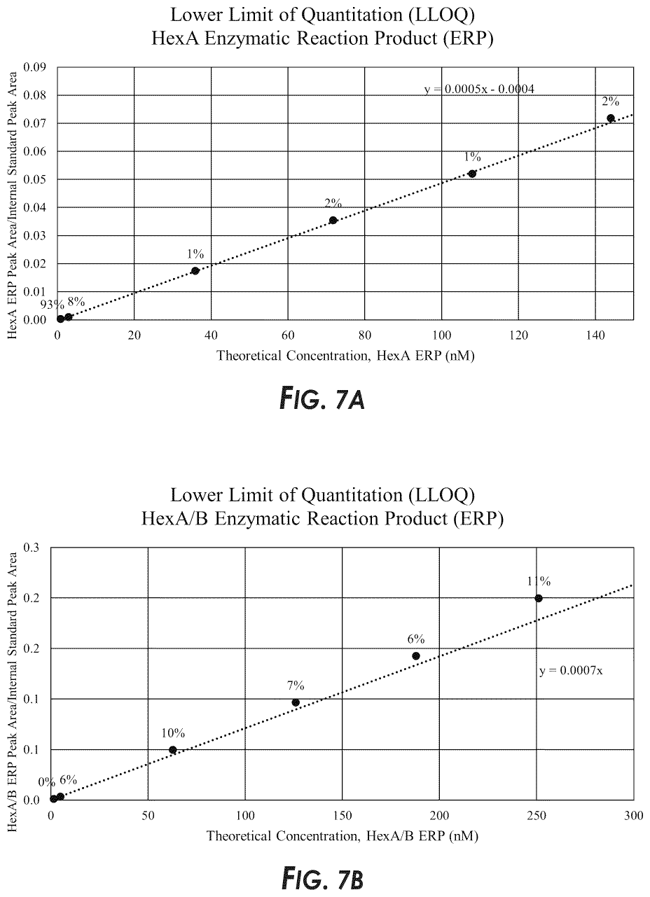

Analytical verification of the assay was completed using the HexA and HexA/B enzymatic reaction products, which were quantified by quantitative nuclear magnetic resonance spectroscopy (qNMR). Linearity of the HexA enzymatic reaction product is shown in FIG. 6A, and linearity of the HexA/B enzymatic reaction product is shown in FIG. 6B. The lower limit of quantification (LLOQ) for the HexA enzymatic reaction product is shown in FIG. 7A, and the LLOQ for the HexA/B enzymatic reaction product is shown in FIG. 7B. Linearity, LLOQ, and intra-assay precision for the HexA and HexA/B enzymatic reaction products are shown in Table 1.

TABLE 1
Analytical verification results

| | HexA enzymatic | | HexA/B enzymatic | |
|---|---|---|---|---|
| Analytical Verification | reaction product | | reaction product | |
| Lower Limit of Quantification | 2.87 | nM | 1.51 | nM |
| (LLOQ)¹ | | | | |
| Max Concentration (Linearity)¹ | 2,530 | nM | 50,000 | nM |
| Intra-assay precision (n = 6)² | 3.7% | CV | 1.7% | CV |
| Intra-assay precision (n = 16)² | 2.8% | CV | 1.2% | CV |

¹The product concentration was analyzed against internal standard across the analytical range of concentrations and was found to be linear (% relative error of line fit for each timepoint is <15%, see FIGS. 6A and 6B) and the LLOQ was determined from six non-zero concentrations (see FIGS. 7A and 7B);
²A single vial of 1:1:1 solution of mixed HexA enzymatic reaction product, HexA/B enzymatic reaction product, and internal standard was prepared (Standard Vial). Intra-assay precision measured as the % coefficient of variation (% CV) within the same LC-MS/MS run for 6 replicate injections of the Standard Vial at the beginning of the run, followed by 10 injections of the Standard Vial interspersed between 10 samples injections throughout the same run. The n = 6 value is the first 6 injections only. The n = 16 value is the first 6 injections plus the following 10 injections. % CV should be below 15%.

### Example 8: Inter-Laboratory Precision of Assay

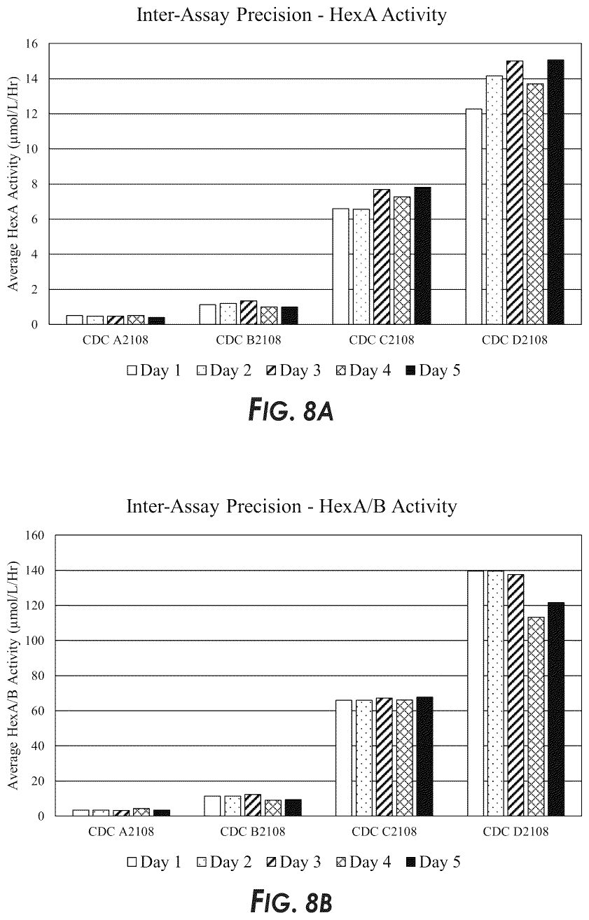

Inter-laboratory precision was tested by two independent laboratories, Lab 1 at the University of Washington (Seattle, Washington, USA) and Lab 2 at ARCHIMEDlife Laboratories (Vienna, Austria, Europe) using a set of four quality control dried blood spots (QCs) from the Center for Disease Control and Prevention (CDC). At Lab 1, the QCs were tested in replicates of n=4 on one day. At Lab 2, the QCs were tested in replicates of n=4 over 4 days. Cross-laboratory inter-assay precision results are shown in Tables 2A and 2B, with graphical representations shown in FIGS. 8A and 8B.

TABLE 2A
Cross-laboratory Precision Results for HexA Activity

| | | HexA Activity [μmol/L/H] Blank corrected | | | | |
|---|---|---|---|---|---|---|
| Condition | | | CDC | CDC | CDC | CDC |
| Day | Laboratory | Blank | A2108 | B2108 | C2108 | D2108 |
| Day 1 | Lab 2 | 0.18 | 0.51 | 1.13 | 6.60 | 12.27 |
| Day 2 | Lab 2 | 1.15 | 0.47 | 1.20 | 6.56 | 14.15 |
| Day 3 | Lab 2 | 1.03 | 0.47 | 1.34 | 7.68 | 15.01 |
| Day 4 | Lab 2 | 0.12 | 0.50 | 0.99 | 7.26 | 13.70 |
| Day 5 | Lab 1 | 0.15 | 0.40 | 1.00 | 7.81 | 15.06 |

| | | | | | |
|---|---|---|---|---|---|
| Average | 0.53 | 0.47 | 1.13 | 7.18 | 14.04 |
| Standard Deviation | 0.518 | 0.044 | 0.145 | 0.587 | 1.144 |
| % CV | 98% | 9% | 13% | 8% | 8% |

TABLE 2B
Cross-laboratory Precision Results for HexA/B Activity

| | | HexA/B Activity [μmol/L/H] Blank corrected | | | | |
|---|---|---|---|---|---|---|
| Condition | | | CDC | CDC | CDC | CDC |
| Day | Laboratory | Blank | A2108 | B2108 | C2108 | D2108 |
| Day 1 | Lab 2 | 0.11 | 3.29 | 10.63 | 62.10 | 122.86 |
| Day 2 | Lab 2 | 0.25 | 3.40 | 11.41 | 65.99 | 139.61 |
| Day 3 | Lab 2 | 0.18 | 3.20 | 12.33 | 67.33 | 137.51 |
| Day 4 | Lab 2 | 0.06 | 4.32 | 9.11 | 66.10 | 113.26 |
| Day 5 | Lab 1 | 0.04 | 3.55 | 9.43 | 67.74 | 121.61 |

| | | | | | |
|---|---|---|---|---|---|
| Average | 0.13 | 3.55 | 10.58 | 65.85 | 126.97 |
| Standard Deviation | 0.090 | 0.448 | 1.347 | 2.231 | 11.230 |
| % CV | 69% | 13% | 13% | 3% | 9% |

### Example 9: Patient Sample Analysis I

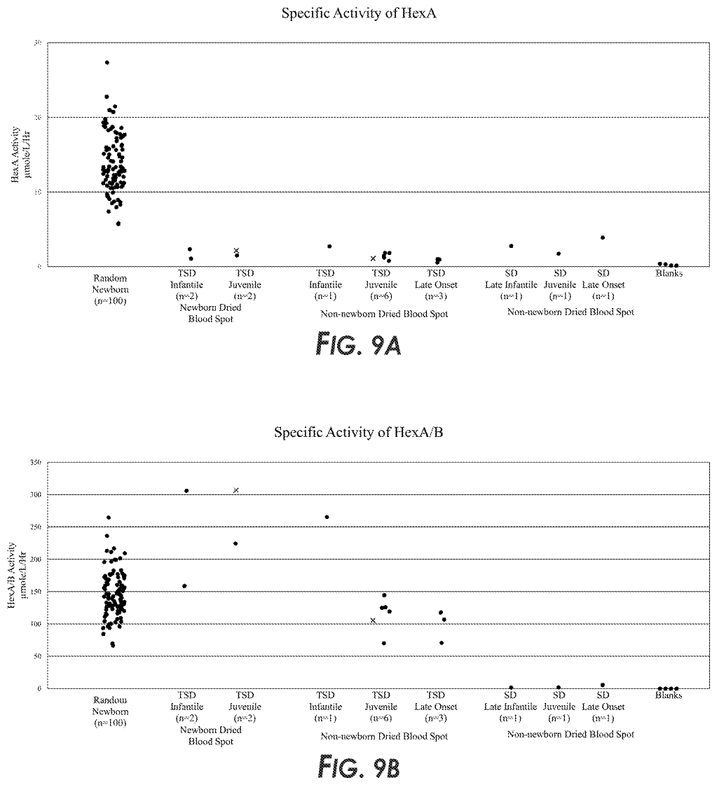

Afflicted newborn dried blood spots (infantile TSD, n=2; and juvenile TSD, n=2) and afflicted non-newborn dried blood spots (infantile TSD, n=1; juvenile TSD, n=6; late onset TSD, n=3; late infantile SD, n=1; juvenile SD, n=1; and late onset SD, n=1) were analyzed for HexA and HexA/B enzymatic activity via measurement of HexA and HexA/B enzymatic reaction products, respectively, using the novel method. Quantities of HexA and HexA/B enzymatic activity from afflicted dried blood spots were compared to a reference range from random non-afflicted newborn dried blood spots (n=100). All samples were tested in the same run on the same day. Assayed HexA and HexA/B enzymatic activity results are shown in Table 3, with graphical representations shown in FIGS. 9A and 9B. Note that two of the dried blood spot samples assayed were from the same afflicted individual and are indicated with an “x” in FIGS. 9A and 9B, whereby the first sample was collected from the newborn patient with juvenile TSD and the second sample was collected from the non-newborn patient with juvenile TSD at age ˜6 years.

TABLE 3
HexA and HexA/B Enzyme Activity in Patient Dried Blood Spots Analyzed at Lab 1

| | | HexA Activity (μmol/L/H) | | HexA/B Activity (μmol/L/H) | |
|---|---|---|---|---|---|
| Patient Status | (n) | Mean (±SD) | Range | Mean (±SD) | Range |
| Random Newborn | 100 | 14.1 (±4.0) | 5.70 − 27.4 | 146 (±36) | 66.6 − 265 |
| TSD Infantile, Newborn | 2 | 1.71 (±0.89) | 1.08 − 2.33 | 232 (±104) | 159 − 306 |
| TSD Juvenile, Newborn | 2 | 1.83 (±0.46) | 1.51 − 2.16 | 266 (±58) | 225 − 307 |
| TSD Infantile, Non-newborn | 1 | 2.73 | — | 266 | — |
| TSD Juvenile, Non-newborn | 6 | 1.37 (±0.43) | 0.791 − 1.86 | 115 (±25) | 70.3 − 145 |
| TSD Late Onset, Non-newborn | 3 | 0.850 (±0.240) | 0.574 − 1.00 | 98.6 (±24.6) | 70.8 − 118 |
| SD Late Infantile, Non-newborn | 1 | 2.77 | — | 1.61 | — |
| SD Juvenile, Non-newborn | 1 | 1.73 | — | 1.90 | — |
| SD Late Onset, Non-newborn | 1 | 3.90 | — | 5.72 | — |

These results show that the dried blood spots from 13 confirmed TSD patients (newborns and non-newborns) displayed less HexA activity than the HexA activity of random non-afflicted newborns, and were also easily discriminated from dried blood spots from three confirmed SD patients by their elevated HexA/B activity levels. The minimum differential, defined as the ratio of the lowest measured activity in the reference range divided by the highest measured activity in the affected samples, was 2.1-fold for confirmed TSD patients (HexA), and the minimum differential for confirmed SD patients was 1.5-fold (HexA) and 12-fold (HexA/B).

### Example 10: Patient Sample Analysis II

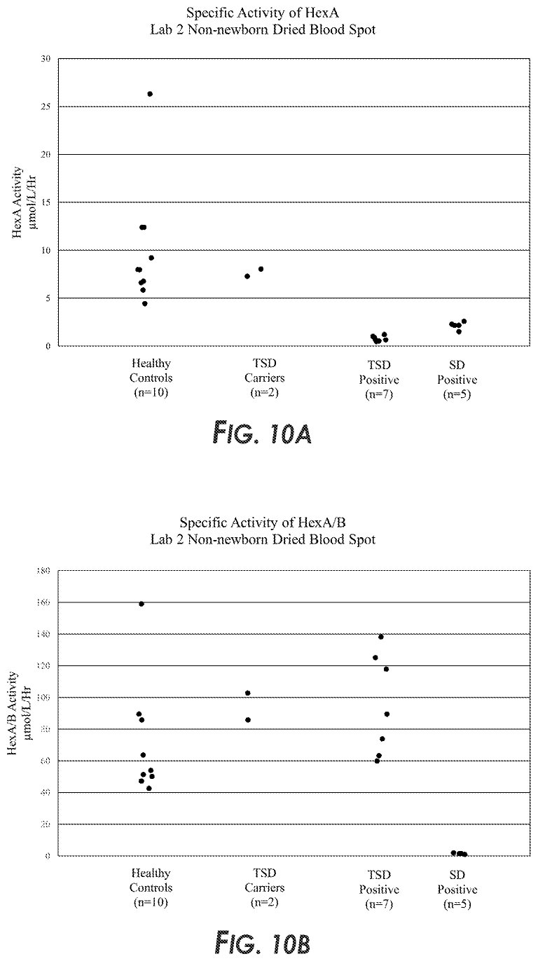

Seven TSD non-newborn dried blood spots and five SD non-newborn dried blood spots were identified by fluorometric assay of HexA and HexA/B during routine screening. These dried blood spots were analyzed for HexA and HexA/B enzymatic activity via measurement of HexA and HexA/B enzymatic reaction products, respectively, using the novel method. In addition to the above, samples from two non-newborn TSD carriers and ten healthy non-newborn controls were concurrently analyzed. Disease status and carrier status were confirmed by genetic analysis. Assayed HexA and HexA/B enzymatic activity results are shown in Table 4, with graphical representations shown in FIGS. 10A and 10B.

TABLE 4
HexA and HexA/B Enzyme Activity in Patient
Dried Blood Spots Analyzed at Lab 2

| | | HexA Activity (μmol/L/H) | | HexA/B Activity (μmol/L/H) | |
|---|---|---|---|---|---|
| Patient Status | (n) | Mean (±SD) | Range | Mean (±SD) | Range |
| Healthy Controls | 10 | 10.0 (±6.3) | 4.4 − 26.0 | 69.2 (±35.6) | 42.7 − 159 |
| TSD Carriers | 2 | 7.67 (±0.55) | 7.29 − 8.06 | 94.4 (±12.0) | 85.9 − 103 |
| TSD Non-newborn | 7 | 0.789 (±0.275) | 0.484 − 1.23 | 95.5 (±31.7) | 60.0− 138 |
| SD Non-newborn | 5 | 2.15 (±0.40) | 1.51 − 2.60 | 1.50 (±0.37) | 0.995 − 2.05 |

### Example 11: Data Interpretation

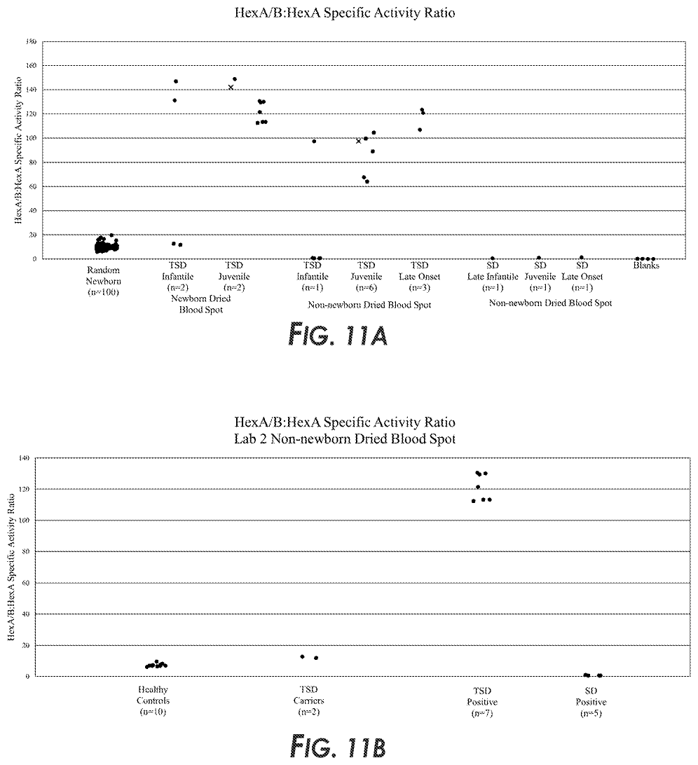

A novel method for data interpretation to compare the specific activity ratio of HexA/B over HexA which may take into consideration a theory that HexB activity may increase in TSD patients to compensate for loss of HexA activity, particularly in infantile TSD, is herein disclosed. Using the “Hex Ratio” (the ratio of HexA/B activity over HexA activity), the minimum differential between confirmed TSD patients and random newborns was found to be increased to 3.3-fold and for SD patients, it was increased to 5.2-fold (shown in FIGS. 11A and 11B).

The above description of particular embodiments of the novel method, its reagents, and its uses is merely exemplary in nature and is not intended to limit the scope of the present invention, its application, or uses, which may, of course, vary. While the steps or compositions may be described as an order of individual steps or using specific materials, it should be appreciated that steps or materials may be interchangeable such that the description of the present invention may include multiple parts or steps arranged in many ways as is readily appreciated by one of skill in the art. Additionally, it should be appreciated that various elements of the above compositions can optionally be substituted for one another.

As can be easily understood from the foregoing, the basic concepts of the present invention may be embodied in a variety of ways. The invention involves numerous and varied embodiments of a method for assaying β-hexosaminidase enzymatic activity and its associated reagents.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “assay” should be understood to encompass disclosure of the act of “assaying”—whether explicitly discussed or not—and, conversely, were there effectively disclosure of the act of “assaying,” such a disclosure should be understood to encompass disclosure of an “assay” and even a “means for assaying.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the methods method for assaying β-hexosaminidase enzymatic activity and its associated reagents herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A method for assaying β-hexosaminidase enzymatic activity, comprising:
combining a sample with a β-hexosaminidase substrate under conditions sufficient for said β-hexosaminidase and said β-hexosaminidase substrate to enzymatically react to produce a β-hexosaminidase enzymatic reaction product; and
detecting said β-hexosaminidase enzymatic reaction product via mass spectrometry.

2. The method of claim 1, wherein said sample comprises a blood sample.

3-11. (canceled)

12. The method of claim 1, further comprising incubating said sample and said β-hexosaminidase substrate at a pre-determined temperature for a pre-determined amount of time sufficient for said β-hexosaminidase and said β-hexosaminidase substrate to enzymatically react to produce said β-hexosaminidase enzymatic reaction product.

13-18. (canceled)

19. The method of claim 1, wherein said β-hexosaminidase substrate is a substrate for β-hexosaminidase A; correspondingly, said β-hexosaminidase substrate comprises a β-hexosaminidase A substrate.

20. The method of claim 19, wherein said β-hexosaminidase A enzymatically reacts with said β-hexosaminidase A substrate to produce a β-hexosaminidase A enzymatic reaction product.

21. The method of claim 20, wherein detection of said β-hexosaminidase A enzymatic reaction product provides a measure of said β-hexosaminidase A activity.

22. The method of claim 20, wherein detection of said β-hexosaminidase A enzymatic reaction product provides a measure of only said β-hexosaminidase A activity.

23. The method of claim 22, wherein β-hexosaminidase B does not enzymatically react with said β-hexosaminidase A substrate.

24. The method of claim 23, wherein enzymatic reaction of said β-hexosaminidase A with said β-hexosaminidase A substrate does not provide a measure of said β-hexosaminidase B activity.

25. The method of claim 1, wherein said β-hexosaminidase substrate is a substrate for β-hexosaminidase A and β-hexosaminidase B; correspondingly, said β-hexosaminidase substrate comprises a β-hexosaminidase A/B substrate.

26. The method of claim 25, wherein said β-hexosaminidase A and said β-hexosaminidase B enzymatically react with said β-hexosaminidase A/B substrate to produce a β-hexosaminidase A/B enzymatic reaction product.

27. The method of claim 26, wherein detection of said β-hexosaminidase A/B enzymatic reaction product provides a measure of said β-hexosaminidase A activity and said β-hexosaminidase B activity.

28. (canceled)

29. The method of claim 1, further comprising performing a quantitative method which measures the amount of said β-hexosaminidase enzymatic reaction product.

30. The method of claim 29, wherein said amount of said β-hexosaminidase enzymatic reaction product is determined by use of an internal standard; and
wherein said internal standard and said β-hexosaminidase enzymatic reaction product have identical chemical structures but are isotopically different.

31-34. (canceled)

35. The method of claim 1, further comprising detecting said β-hexosaminidase enzymatic reaction product via tandem mass spectrometry.

36. The method of claim 1, further comprising combining said sample with more than one β-hexosaminidase substrate to correspondingly produce more than one said β-hexosaminidase enzymatic reaction product; and
detecting said β-hexosaminidase enzymatic reaction products via said mass spectrometry.

37. The method of claim 36, wherein a first said β-hexosaminidase substrate comprises a β-hexosaminidase A substrate with which β-hexosaminidase A enzymatically reacts to produce a β-hexosaminidase A enzymatic reaction product; and
wherein a second said β-hexosaminidase substrate comprises a β-hexosaminidase A/B substrate with which said β-hexosaminidase A and β-hexosaminidase B enzymatically react to produce a β-hexosaminidase A/B enzymatic reaction product.

38-50. (canceled)

51. A method for screening an individual for a deficiency in β-hexosaminidase A enzymatic activity, comprising:
combining a sample from said individual with a β-hexosaminidase A substrate under conditions sufficient for said β-hexosaminidase A and said β-hexosaminidase A substrate to enzymatically react to produce a β-hexosaminidase A enzymatic reaction product; and
detecting the presence of said β-hexosaminidase A enzymatic reaction product via mass spectrometry;
wherein the absence of said β-hexosaminidase A enzymatic reaction product indicates said deficiency in said β-hexosaminidase A enzymatic activity.

52. The method of claim 51, wherein said deficiency in said β-hexosaminidase A enzymatic activity indicates that said individual may be diagnosed with Tay-Sachs disease.

53. The method of claim 51, wherein said deficiency in said β-hexosaminidase A enzymatic activity indicates that said individual may be a candidate for treatment for Tay-Sachs disease.

54-79. (canceled)
